SEQUENCE LISTING (16 sequences)

SEQ ID NO: 1 - protein, 242 aa, Homo sapiens
  REGION 1..242: note = 14-3-3 beta protein; brain protein 14-3-3 beta isoform
  REGION 1..242: note = Alternate nomenclature: Tyrosine 3-monooxygenase/ tryptophan 5-monooxygenase activation protein, beta isoform, YWHAB
MTMDKSELVQKAKLAEQAERYDDMAAAMKAVTEQGHELSNEERNLLSVAYKNVVGARRSSWRVISSIEQKTERNEKKQQMGKEYREKIEAELQDICNDVLELLDKYLIPNATQPESKVFYLKMKGDYFRYLSEVASGDNKQTTVSNSQQAYQEAFEISKKEMQPTHPIRLGLALNFSVFYYEILNSPEKACSLAKTAFDEAIAELDTLNEESYKDSTLIMQLLRDNLTLWTSENQGDEGDAG

SEQ ID NO: 2 - protein, 246 aa, Homo sapiens
  REGION 1..246: note = 14-3-3 gamma protein
  REGION 1..246: note = Alternate nomenclature: Tyrosine 3-monooxygenase/ tryptophan 5-monooxygenase activation protein, gamma isoform, YWHAG
MVDPEQLVQKARLAEQAEGYDDMAAAMKNVTELNEPLSNEERNLLSVAYKNVVGARRSSWRVISSIEQKTSADGNEKIEMVRAYREKVEKELEAVCQDVLSLVDNYLYKNCSETQYERKDLYLKMKGDYYRYLAEVATGEKRGDVVESSEKAYSEREISKEHMQPTHPIRLGLALNYSVFYYEIQNAPEQACHLAKTEFEDAIAELDTLNEESYKDSTLIMQLLRDNLTLWTSDQQDDHDGGEGNN

SEQ ID NO: 3 - protein, 244 aa, Homo sapiens
  REGION 1..244: note = 14-3-3 eta protein
  REGION 1..244: note = Alternate nomenclature: Tyrosine 3-monooxygenase/ tryptophan 5-monooxygenase activation protein, eta isoform, YWHAH;
  REGION 1..244: note = brain protein 14-3-3, eta isoform tyrosine 3-monooxygenase/tryptophan 5-monooxygenase activation protein 1; YWHA1
MGDREQLLQRARLAEQAERYDDMASAMKAVTELNEPLSNEDRNLLSVAYKNVVGARRSSWRVISSIEQKTMADGNEKKLEKVKAYREKIEKELETVCNDVLSLLDKFLIKNCNDFQYESKVFYLKMKGDYYRYLAEVASGEKKNSVVEASEAAYKEAFEISKEQMQPTHPIRLGLALNFSVFYYEIQNAPEQACLLAKQAFDDAIAELDTLNEDSYKDSTLIMQLLRDNLTLWTSDQQDEEAGE

SEQ ID NO: 4 - protein, 255 aa, Homo sapiens
  REGION 1..255: note = 14-3-3 epsilon protein
  REGION 1..255: note = Alternate nomenclature: Tyrosine 3-monooxygenase/ tryptophan 5-monooxygenase activation protein, epsilon isoform, YWHAE epsilon isoform
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 5 - protein, 247 aa, Homo sapiens
  REGION 1..247: note = 14-3-3 sigma protein
  REGION 1..247: note = Alternate nomenclature: stratifin
MERASLIQKAKLAEQAERYEDMAAFMKGAVEKGEELSCEERNLLSVAYKNVVGGQRAAWRVLSSIEQKSNEEGSEEKGPEVREYREKVETELQGVCDTVLGLLDSHLIKEAGDAESRVFYLKMKGDYYRYLAEVATGDDKKRIIDSARSAYQEAMDISKKEMPPTNPIRLGLALNFSVFHYEIANSPEEAISLAKTTFDEAMADLHTLSEDSYKDSTLIMQLLRDNLTLWTADNAGEEGGEAPQEPQ

SEQ ID NO: 6 - protein, 245 aa, Homo sapiens
  REGION 1..245: note = 14-3-3 tau protein
  REGION 1..245: note = Alternate nomenclature: Tyrosine 3-monooxygenase/ tryptophan 5-monooxygenase activation protein, tau isoform, YWHAQ 14-3-3 protein, T-cell 14-3-3-theta HS1
MEKTELIQKAKLAEQAERYDDMATCMKAVTEQGAELSNEERNLLSVAYKNVVGGRRSAWRVISSIEQKTDTSDKKLQLIKDYREKVESELRSICTTVLELLDKYLIANATNPESKVFYLKMKGDYFRYLAEVACGDDRKQTIDNSQGAYQEAFDISKKEMQPTHPIRLGLALNFSVFYYEILNNPELACTLAKTAFDEAIAELDTLNEDSYKDSTLIMQLLRDNLTLWTSDSAGEECDAAEGAEN

SEQ ID NO: 7 - protein, 245 aa, Homo sapiens
  REGION 1..245: note = 14-3-3 zeta protein
  REGION 1..245: note = Alternate nomenclature: Tyrosine 3-monooxygenase/ tryptophan 5-monooxygenase activation protein, zeta isoform, YWHAZ; brain protein 14-3-3, zeta isoform
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 8 - DNA, 3015 bp, Homo sapiens
  misc_feature 1..3015: note = 14-3-3 beta cDNA (NM_139323) 3015 nt
  source 1..3015: mol_type = genomic DNA
gcggaagtggagctaccgccaccgccgccgccgattccggagccggggtagtcgccgccgccgccgccgctgcagccactgcaggcaccgctgccgccgcctgagtagtgggcttaggaaggaagaggtcatctcgctcggagcttcgctcggaagggtctttgttccctgcagccctcccacgggaatgacaatggataaaagtgagctggtacagaaagccaaactcgctgagcaggctgagcgatatgatgatatggctgcagccatgaaggcagtcacagaacaggggcatgaactctccaacgaagagagaaatctgctctctgttgcctacaagaatgtggtaggcgcccgccgctcttcctggcgtgtcatctccagcattgagcagaaaacagagaggaatgagaagaagcagcagatgggcaaagagtaccgtgagaagatagaggcagaactgcaggacatctgcaatgatgttctggagctgttggacaaatatcttattcccaatgctacacaaccagaaagtaaggtgttctacttgaaaatgaaaggagattattttaggtatctttctgaagtggcatctggagacaacaaacaaaccactgtgtcgaactcccagcaggcttaccaggaagcatttgaaattagtaagaaagaaatgcagcctacacacccaattcgtcttggtctggcactaaatttctcagtcttttactatgagattctaaactctcctgaaaaggcctgtagcctggcaaaaacggcatttgatgaagcaattgctgaattggatacgctgaatgaagagtcttataaagacagcactctgatcatgcagttacttagggacaatctcactctgtggacatcggaaaaccagggagacgaaggagacgctggggagggagagaactaatgtttctcgtgctttgtgatctgttcagtgtcactctgtaccctcaacatatatcccttgtgcgataaaaaaaaaaaaaaaaaaaaaaagagaatcgtacgtcgactttcgatttttcacagcctcagcctaggaaaaatggttcatgggataaacagctggtatttgtatctaaaactcagattggtcacataaatgccacggcattccgaagttttgattttgattaacattgacaggattactgtgtgtttaattttttaaaaactgaacactgtgattatggggttttgtaatttagcagaactcttactggtagaaaaaatagacctgaattatgtgtaactttttggaaggtttaatctgatatcaaaataatcattgaaatacaattccattgtaaagttgtacagaaagttatagagattatattgtgatgctggaacttggagtgagacacacatcatttggcatttgagttgaatggtaattcacagtaatgctgccgttgttcgggacttaaagacacttgacctgtttgggctgttgccacttaaaagttcatgaccacaaatgtccacagtgtcttcctctgaggaaactcgaatcctgaaatggaaattctttgtggcagataactggcttatgacaccttgaaaagttcaagtgctcatataacacaccacactgaaccccctttcctacagcaatatgttcactatgttaccaatttgcaacttgtgcttcaatagtggaatctactttcattgttaacactgagctaaagaaaaaaagccgtgtgttttatgaatgaccttatctgtttcctggataatacctttaagaataatgtcctgagtcaggcgtggtggtgcgtgcatctagtcccaactatttgggaggctgaggcaggaggatcgcttgagcccaggagtttaaagctgcagtgccctgtggttgcacctgtgaataactgcactccagcctgggcaacatagcgagacctcatctccaaaaaagaaaacaaaaaacaaaaaaaggaatgatgttctgtagagatggcctttcacttgaggagtactcagttttcaggttcttcctagctcggggcttttaaattttgaaatctaaacattctttcccaccatcctttttgactgttgaccttggttttctcttctaagtttctgtccctctgcttccttactttttttcctttttgaattctatctttatctgtcttttgttcactttttaatgctatatatgggcaggggtgagagacattactgagcaccttggtgagcaagcctggctttaaagattggagaagagcttctggcaccagaaccctgtcttcctccagttctcaacacggtgttgctcttcagtcataccggaatctgaatcaaaaaagtatttttaaatatccatgatttctccctgtattgaggctagccctgatcatgctttttgtgcctgtcaccaggtctcccaagtgcactcatccaggtcagtgctcagatgtgtttaaggagaccctatattcagggaagttgcgtgaacactgcagtggggagaattgagaatagtcaggcctatcagtctcacagaatcacccctctacctttgatattccacttagctgtagagtccatctgtttgtccatctgctgaaatgagaaaagaaaaatttatgcactgatttaaaacaaaccaaaaaaaaagaaaaaaacaaaaaaaaaaatccctcctttctagctgaacaaaaatgtgcagttaatacttggcgcttgaaaatgcagtagtgaatgtggaaccaagcctgtctgtatatctggtagctcttttcttgctttgttttttcttaccagtattctgcctaacgtttgcttctgtgatggttatattgcctagcaagcacacccgtggttgtgaaaatagtatagcaaaaaagaaaaatccccggttattgatgtactagatttgtgtatgtcttttaaacagttctagtttcaccttacacagaataatcaggaaaagtgtaaaaattcaaaagtgaaataaaaattttatcagttaaaaaa

SEQ ID NO: 9 - DNA, 3747 bp, Homo sapiens
  misc_feature 1..3747: note = 14-3-3 gamma cDNA (NM_012479) 3747 nt
  source 1..3747: mol_type = genomic DNA
cagcagccgcagccgcctcgcgcccggtcccgcggtcgcagctccagccgcctcctccgcgcagccgccgcctcagctgctcgctctgtgggtcggtcctctccggcacttgggctccagtcgcgccctccaagcccttcaggccgccccagtgtcctcctccttctccggccagacccagccccgcgaagatggtggaccgcgagcaactggtgcagaaagcccggctggccgagcaggcggagcgctacgacgacatggccgcggccatgaagaacgtgacagagctgaatgagccactgtcgaatgaggaacgaaaccttctgtctgtggcctacaagaacgttgtgggggcacgccgctcttcctggagggtcatcagtagcattgagcagaagacatctgcagacggcaatgagaagaagattgagatggtccgtgcgtaccgggagaagatagagaaggagttggaggctgtgtgccaggatgtgctgagcctgctggataactacctgatcaagaattgcagcgagacccagtacgagagcaaagtgttctacctgaagatgaaaggggactactaccgctacctggctgaagtggccaccggagagaaaagggcgacggtggtggagtcctccgagaaggcctacagcgaagcccacgagatcagcaaagagcacatgcagcccacccaccccatccgattaggcctggctcttaactactccgtcttctactatgagatccagaacgccccagagcaagcgtgccacttggccaagaccgcgttcgacgacgccatcgccgagcttgacaccctcaacgaggactcctacaaggactccacgctcatcatgcagctcctccgcgacaacctcacgctctggacgagcgaccagcaggacgacgatggcggcgaaggcaacaattaaggccccaggggaactggcagcgcacgcggatgctactactgcagtctttatttttttcccatgagttgggggtcgggtgggggagggaaagggagggatgaccttcccagggagaaacccacgacctgtcctgtctttgatcgcctctttgacatttttgccaaaataccactagtggaaagtcaggctagctgtgctggtattggaatagcagcctcacactggcgtctggactgttctgtagattcatgcaagtggagctgtctgtctctaatttaacttattgctagataatagggttttcagatgaaaagaaaacttaaagaggaatggccctcattcagtaagttctgtggttccagtaaggatttttatgtacatacgctctcgtctctcgttttgggtactttctatctcatctgtctcggctctgcatgttttccagggtgtagcctacagacatggaacagtgtaaatcccagactgacagacttagaacctgaggtctcattcatccttatggtttaggccttgccagttttccgaagtctctgattagttgacagtattaacactaaattgcagtttacagtatttctacattacagccatatgtaacatcaagccatcgattgtgtacttttcctttgctagttgtttgggctttaacatccttattcagccttatccaggttggttttgctgttgatcggtctcctaggctaaatgagaatgaaagcgacttcaggtttttggttcataggtgctcggcaggtggctgtgggattttttttttttttggtccttctttcctcttaacgtaaatccaccaccaaaattattaatcctcttgagagaaacgtgaaacgccacaaaaatagagaaaattcaggtctgtatgtcatggatcgtgttggtattttcagagaacatcccgcttctgaagctgctgcagctccctcctcagggatcacactgccgtcacccactctgcactggggcgtttcctactgcgcctcgtgctggcggacgcagctgggtgcagaagctgtggggtcggagaggcgtttggagaaggtctgtggtgcagtgtgtgaaaattcaggtgctagaagcctactggtagaaaaacccaaaaggaagagctatatccttaaccattctgtccaatttcgggagccttgtcagtgtgtcagtttttcctccccgaagacactccttccccaagtaattgtaggaagataaaaaaactgttaccagataacaaacactgaactcctatttgaccagaactttttcctctcgagatagttttttctttttaatgaaaaaagcataggaattggagattggcttgtctcacgcagccagtgcacatttggaattgacggaaacaacgttgctatttccacccatttgttttcggcagccttaaggccctcattctcatttcgggtgaatctgtctatctgtgaacgtggcccgcatgtgcattcttttttttatatatataaagtcagtgacgaggaactcccgagacgtgtaatgacaccacacttgttttctttgtttctttgttttatttaggcaagaagaggtgtgagtaattgaggaaaaactgacagatgcttttgctaataccaaaattgagcttacaattaggaactgagtatgtgtaacaggatacaggtgacagtgaagatagaagaaccacgatgaccacagactcaatgtgctctgtaacatcgcacagtttacccagcatgactttccttaggaggccccctcctcacgctagagtaaaagtcccagttaagtgaagcctaccagaagaactagtagaagaagctttgccgcttttgtgcctctcacaggcgcctaaagtcattgccatgggaggaagacgatttggggggggagggggggggcagggtaggtggggctttccctaatttatcttcatgtccagtgagcagtgttgcgtttttccttgtagcatttggaaatgatttactggaattacaaaacctatttttcctttaaatttcagctttggctctggctgctttttagaataatgcaagataaaaatcacacctgagggctgaaaacggagagggaatgggagacttgatatttaagcagcttgaatggtttttcttttctttatttttaaagaaatgcacttgcctatgatactgtctctccagtgaaatgattactcctccattactctattgatacaatattgtgcatgctagtgttgtatttctatacagtagcttgaaattgattaacttatactgtaggtgttatgtattcctatgacaaaaaaaattaagtcttcaaattttttaaaggttttttttttttaatttaatttttccttttgggggtaaagtttgctctaccaaatagtgattgtaacaaattgatctgttttggatgttgctatagtgacatgcagttatatattttgtttttaaaagggggggagcaaaagaaacaccagtgttagcttaatcttaatgtctggtgtttgtcatggtgaaattataactattacagtgttggagaacaacaaatatgttctctgaatgagcctttgtgctttttgtcatgttatgcagtgaactatttttaaggtctaatcagtgattatttttccagctccgtgtttctctaaggaattatttcacacacggaccatctttagcagtttcctcagtgatggaatatcatgaatgtgagtcattatgtagctgtcgtacattgagcaaataaacttacagatctgaaaaaaaaaaaaaaa

SEQ ID NO: 10 - DNA, 1810 bp, Homo sapiens
  misc_feature 1..1810: note = 14-3-3 epsilon cDNA (NM_006761) 1810 nt
  source 1..1810: mol_type = genomic DNA
cggattgaggcgccgccatttttgctgcccggacgcggagcgagaggctgagagagtcggagacactatccgcttccatccgtcgcgcagaccctgccggagccgctgccgctatggatgatcgagaggatctggtgtaccaggcgaagctggccgagcaggctgagcgatacgacgaaatggtggagtcaatgaagaaagtagcagggatggatgtggagctgacagttgaagaaagaaacctcctatctgttgcatataagaatgtgattggagctagaagagcctcctggagaataatcagcagcattgaacagaaagaagaaaacaagggaggagaagacaagctaaaaatgattcgggaatatcggcaaatggttgagactgagctaaagttaatctgttgtgacattctggatgtactggacaaacacctcattccagcagctaacactggcgagtccaaggttttctattataaaatgaaaggggactaccacaggtatctggcagaatttgccacaggaaacgacaggaaggaggctgcggagaacagcctagtggcttataaagctgctagtgatattgcaatgacagaacttccaccaacgcatcctattcgcttaggtcttgctctcaatttttccgtattctactacgaaattcttaattcccctgaccgtgcctgcaggttggcaaaagcagcttttgatgatgcaattgcagaactggatacgctgagtgaagaaagctataaggactctacacttatcatgcagttgttacgtgataatctgacactatggacttcagacatgcagggtgacggtgaagagcagaataaagaagcgctgcaggacgtggaagacgaaaatcagtgagacataagccaacaagagaaaccatctctgaccaccccctcctccccatcccaccctttggaaactccccattgtcactgagaaccaccaaatctgacttttacatttggtctcagaatttaggttcctgccctgttggttttttttttttttttttttaaacagttttcaaaagttcttaaaggcaagagtgaatttctgtggattttactggtcccagcttttaggttctttaagacactaacaggactacatagaggctttttcagcattactgtgtcgtctccgtgccagatgtggcaagatcaccattagcaaatggaaattacatttgaaagccattagacttataggtgatgcaagcatctaagagagaggttaatcacactatagaggcataagtggtatcagttttcatttttctaattgtttaaactgtgttttataccagtgtttgcaagtaattgggtgttagcttgagatggttaaaggtggtttggggagggacttcgttgtaatggttttgctgtaaaaaatgtttccaactccgctgaaatgttgctgaaaagcatggtgctggtaacagttcaacaatccgtggctgctcattcttgcctactttactctcccactgaagcaggttagcgttgaaggtggtatggaaaagcctgcatgcctgttcaattcttttgtttcttctccttccccctccccctacctccttcccctcactcctcccctccttcgctcgctcaacctcttttgttcagtatgtgtaacttgaagctaatttgtactactggatatctgactggagccacagatacagaatctgtattgttcttactgaaacacagcatggaattaacattaaacttaaataaaacaaacctaaattaaaaaaaaaaaaaaaaaaaaaaaaaaaaaaa

SEQ ID NO: 11 - DNA, 1807 bp, Homo sapiens
  misc_feature 1..1807: note = 14-3-3 eta cDNA (NM_003405) 1807 nt
  source 1..1807: mol_type = genomic DNA
gcggccgcgtctcctccctcggcgttgtccgcggcgcgagccacagcgcgcggggcgagccagcgagagggcgcgagcggcggcgctgcctgcagcctgcagcctgcagcctccggccggccggcgagccagtgcgcgtgcgcggcggcggcctccgcagcgaccggggagcggactgaccggcgggagggctagcgagccagcggtgtgaggcgcgaggcgaggccgagccgcgagcgacatgggggaccgggagcagctgctgcagcgggcgcggctggccgagcaggcggagcgctacgacgacatggcctccgctatgaaggcggtgacagagctgaatgaacctctctccaatgaagatcgaaatctcctctctgtggcctacaagaatgtggttggtgccaggcgatcttcctggagggtcattagcagcattgagcagaaaaccatggctgatggaaacgaaaagaaattggagaaagttaaagcttaccgggagaagattgagaaggagctggagacagtttgcaatgatgtcctgtctctgcttgacaagttcctgatcaagaactgcaatgatttccagtatgagagcaaggtgttttacctgaaaatgaagggtgattactaccgctacttagcagaggtcgcttctggggagaagaaaaacagtgtggtcgaagcttctgaagctgcctacaaggaagcctttgaaatcagcaaagagcagatgcaacccacgcatcccatccggctgggcctggccctcaacttctccgtgttctactatgagatccagaatgcacctgagcaagcctgcctcttagccaaacaagccttcgatgatgccatagctgagctggacacactaaacgaggattcctataaggactccacgctgatcatgcagttgctgcgagacaacctcaccctctggacgagcgaccagcaggatgaagaagcaggagaaggcaactgaagatccttcaggtcccctggcccttccttcacccaccacccccatcatcaccgattcttccttgccacaatcactaaatatctagtgctaaacctatctgtattggcagcacagctactcagatctgcactcctgtctcttgggaagcagtttcagataaatcatgggcattgctggactgatggttgctttgagcccacaggagctccctttttgaattgtgtggagaagtgtgttctgatgaggcattttactatgcctgttgatctatgggaaatctaggcgaaagtaatggggaagattagaaagaattagccaaccaggctacagttgatatttaaaagatccatttaaaacaagctgatagtgtttcgttaagcagtacatcttgtgcatgcaaaaatgaattcacccctcccacctctttcttcaattaatggaaaactgttaagggaagctgatacagagagacaacttgctcctttccatcagctttataataaactgtttaacgtgaggtttcagtagctccttggttttgcctctttaaattatgacgtgcacaaaccttcttttcaatgcaatgcatctgaaagttttgatacttgtaacttttttttttttttggttgcaattgtttaagaatcatggatttattttttgtaactctttggctattgtccttgtgtatcctgacagcgccatgtgtgtcagcccatgtcaatcaagatgggtgattatgaaatgccagacttctaaaataaatgttttggaattcaatgggtaaataaatgctgctttggggatattaaaaaaaaaaaaaaa

SEQ ID NO: 12 - DNA, 2166 bp, Homo sapiens
  misc_feature 1..2166: note = 14-3-3 tau cDNA (NM_006826) 2166 nt
  source 1..2166: mol_type = genomic DNA
gtggtgggactcgcgtcgcggccgcggagacgtgaagctctcgaggctcctcccgctgcgggtcggcgctcgccctcgctctcctcgccctccgccccggccccggccccgcgcccgccatggagaagactgagctgatccagaaggccaagctggccgagcaggccgagcgctacgacgacatggccacctgcatgaaggcagtgaccgagcagggcgccgagctgtccaacgaggagcgcaacctgctctccgtggcctacaagaacgtggtcgggggccgcaggtccgcctggagggtcatctctagcatcgagcagaagaccgacacctccgacaagaagttgcagctgattaaggactatcgggagaaagtggagtccgagctgagatccatctgcaccacggtgctggaattgttggataaatatttaatagccaatgcaactaatccagagagtaaggtcttctatctgaaaatgaagggtgattacttccggtaccttgctgaagttgcgtgtggtgatgatcgaaaacaaacgatagataattcccaaggagcttaccaagaggcatttgatataagcaagaaagagatgcaacccacacacccaatccgcctggggcttgctcttaacttttctgtattttactatgagattcttaataacccagagcttgcctgcacgctggctaaaacggcttttgatgaggccattgctgaacttgatacactgaatgaagactcatacaaagacagcaccctcatcatgcagttgcttagagacaacctaacactttggacatcagacagtgcaggagaagaatgtgatgcggcagaaggggctgaaaactaaatccatacagggtgtcatccttctttccttcaagaaacctttttacacatctccattccttattccacttggatttcctatagcaaagaaacccattcatgtgtatggaatcaactgtttatagtcttttcacactgcagctttgggaaaacttcattccttgatttgtgtttgtcttggccttcctggtgtgcagtactgctgtagaaaagtattaatagcttcatttcatataaacataagtaactcccaaacacttatgtagaggactaaaaatgtatctggtatttaagtaatctgaaccagttctgcaagtgactgtgttttgtattactgtgaaaataagaaaatgtagttaattacaatttaaagagtattccacataacttcttaatttctacattccctcccttactcttcgggggtttcctttcagtaagcaacttttccatgctcttaatgtattcctttttagtaggaatccggaagtattagattgaatggaaaagcacttgccatctctgtctaggggtcacaaattgaaatggctcctgtatcacatacggaggtcttgtgtatctgtggcaacagggagtttccttattcactctttatttgctgctgtttaagttgccaacctcccctcccaataaaaattcacttacacctcctgcctttgtagttctggtattcactttactatgtgatagaagtagcatgttgctgccagaatacaagcattgcttttggcaaattaaagtgcatgtcatttcttaatacactagaaaggggaaataaattaaagtacacaagtccaagtctaaaactttagtacttttccatgcagatttgtgcacatgtgagagggtgtccagtttgtctagtgattgttatttagagagttggaccactattgtgtgttgctaatcattgactgtagtcccaaaaaagccttgtgaaaatgttatgccctatgtaacagcagagtaacataaaataaaagtacattttataaaccatttactatggctttgtaacaattgcatacccatattttaagggacaggtgaatttactactttctaaagtttattgatacttcccttttatgtaaaatgtagtagtgatacctatatttccacattgtgcattgtgacacacttgtctagggatgcctggaagtgtataaaattggactgcatttcttagagtgttttactatagatcagtctcatgggccatctcttcctcagatgtaaatgatatctggttaagtgttatatggaataaagtggacattttaaaacta

SEQ ID NO: 13 - DNA, 1329 bp, Homo sapiens
  misc_feature 1..1329: note = 14-3-3 sigma cDNA (NM_006142) 1336 nt
  source 1..1329: mol_type = genomic DNA
gagagacacagagtccggcattggtcccaggcagcagttagcccgccgcccgcctgtgtgtccccagagccatggagagagccagtctgatccagaaggccaagctggcagagcaggccgaacgctatgaggacatggcagccttcatgaaaggcgccgtggagaagggcgaggagctctcctgcgaagagcgaaacctgctctcagtagcctataagaacgtggtgggcggccagagggctgcctggagggtgctgtccagtattgagcagaaaagcaacgaggagggctcggaggagaaggggcccgaggtgcgtgagtaccgggagaaggtggagactgagctccagggcgtgtgcgacaccgtgctgggcctgctggacagccacctcatcaaggaggccggggacgccgagagccgggtcttctacctgaagatgaagggtgactactaccgctacctggccgaggtggccaccggtgacgacaagaagcgcatcattgactcagcccggtcagcctaccaggaggccatggacatcagcaagaaggagatgccgcccaccaaccccatccgcctgggcctggccctgaacttttccgtcttccactacgagatcgccaacagccccgaggaggccatctctctggccaagaccactttcgacgaggccatggctgatctgcacaccctcagcgaggactcctacaaagacagcaccctcatcatgcagctgctgcgagacaacctgacactgtggacggccgacaacgccggggaagaggggggcgaggctccccaggagccccagagctgagtgttgcccgccaccgccccgccctgccccctccagtcccccaccctgccgagaggactagtatggggtgggaggccccacccttctcccctaggcgctgttcttgctccaaagggctccgtggagagggactggcagagctgaggccacctggggctggggatcccactcttcttgcagctgttgagcgcacctaaccactggtcatgcccccacccctgctctccgcacccgcttcctcccgaccccaggaccaggctacttctcccctcctcttgcctccctcctgcccctgctgcctctgatcgtaggaattgaggagtgtcccgccttgtggctgagaactggacagtggcaggggctggagatgggtgtgtgtgtgtgtgtgtgtgtgtgtgtgtgtgtgcgcgcgcgccagtgcaagaccgagattgagggaaagcatgtctgctgggtgtgaccatgtttcctctcaataaagttcccctgtgacactcaaaaaaaaaaaaaa

SEQ ID NO: 14 - DNA, 2876 bp, Homo sapiens
  misc_feature 1..2876: note = 14-3-3 zeta cDNA (NM_145690) 2876 nt
  source 1..2876: mol_type = genomic DNA
gcctgtgagcagcgagatccagggacagagtctcagcctcgccgctgctgccgccgccgccgcccagagactgctgagcccgtccgtccgccgccaccacccactccggacacagaacatccagtcatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggatacccaaggagacgaagctgaagcaggagaaggaggggaaaattaaccggccttccaacttttgtctgcctcattctaaaatttacacagtagaccatttgtcatccatgctgtcccacaaatagttttttgtttacgatttatgacaggtttatgttacttctatttgaatttctatatttcccatgtggtttttatgtttaatattaggggagtagagccagttaacatttagggagttatctgttttcatcttgaggtggccaatatggggatgtggaatttttatacaagttataagtgtttggcatagtacttttggtacattgtggcttcaaaagggccagtgtaaaactgcttccatgtctaagcaaagaaaactgcctacatactggtttgtcctggcggggaataaaagggatcattggttccagtcacaggtgtagtaattgtgggtactttaaggtttggagcacttacaaggctgtggtagaatcataccccatggataccacatattaaaccatgtatatctgtggaatactcaatgtgtacacctttgactacagctgcagaagtgttcctttagacaaagttgtgacccattttactctggataagggcagaaacggttcacattccattatttgtaaagttacctgctgttagctttcattatttttgctacactcattttatttgtatttaaatgttttaggcaacctaagaacaaatgtaaaagtaaagatgcaggaaaaatgaattgcttggtattcattacttcatgtatatcaagcacagcagtaaaacaaaaacccatgtatttaacttttttttaggatttttgcttttgtgattttttttttttttttttgatacttgcctaacatgcatgtgctgtaaaaatagttaacagggaaataacttgagatgatggctagctttgtttaatgtcttatgaaattttcatgaacaatccaagcataattgttaagaacacgtgtattaaattcatgtaagtggaataaaagttttatgaatggacttttcaactactttctctacagcttttcatgtaaattagtcttggttctgaaacttctctaaaggaaattgtacattttttgaaatttattccttattccctcttggcagctaatgggctcttaccaagtttaaacacaaaatttatcataacaaaaatactactaatataactactgtttccatgtcccatgatcccctctcttcctccccaccctgaaaaaaatgagttcctattttttctgggagagggggggattgattagaaaaaaatgtagtgtgttccatttaaaattttggcatatggcattttctaacttaggaagccacaatgttcttggcccatcatgacattgggtagcattaactgtaagttttgtgcttccaaatcactttttggtttttaagaatttcttgatactcttatagcctgccttcaattttgatcctttattctttctatttgtcaggtgcacaagattaccttcctgttttagccttctgtcttgtcaccaaccattcttacttggtggccatgtacttggaaaaaggccgcatgatctttctggctccactcagtgtctaaggcaccctgcttcctttgcttgcatcccacagactatttccctcatcctatttactgcagcaaatctctccttagttgatgagactgtgtttatctccctttaaaaccctacctatcctgaatggtctgtcattgtctgcctttaaaatccttcctctttcttcctcctctattctctaaataatgatggggctaagttatacccaaagctcactttacaaaatatttcctcagtactttgcagaaaacaccaaacaaaaatgccattttaaaaaaggtgtattttttcttttagaatgtaagctcctcaagagcagggacaatgttttctgtatgttctattgtgcctagtacactgtaaatgctcaataaatattgatgatgggaggcagtgagtcttgatgataagggtgagaaactgaaatccc

SEQ ID NO: 15 - DNA, 31 bp, synthetic construct
  misc_feature 1..31: note = Description of Artificial Sequence: Synthetic primer used for PCR amplification of the 14-3-3 eta cDNA
  source 1..31: mol_type = other DNA
gcgaattcctgcagcgggcgcggctggccga

SEQ ID NO: 16 - DNA, 31 bp, synthetic construct
  misc_feature 1..31: note = Description of Artificial Sequence: Synthetic primer used for PCR amplification of the 14-3-3 eta cDNA
  source 1..31: mol_type = other DNA
gctcgagcctgaaggatcttcagttgccttc

(19) United States
(12) Patent Application Publication
(10) Pub. No.: US 2023/0366892 A1
(43) Pub. Date: Nov. 16, 2023

(54) Dissolved Protein Arthritis Markers

(71) Applicant: The University of British Columbia, Vancouver (CA)

(72) Inventors: Aziz GHAHARY, Vancouver (CA); Wolodymyr Walter Peter MAKSYMOWYCH, Edmonton (CA); Ruhangiz Taghi KILANI, Vancouver (CA)

(21) Appl. No.: 18/212,115

(22) Filed: Jun. 20, 2023

Related U.S. Application Data
(63) Continuation of application No. 16/868,447, filed on May 6, 2020, now Pat. No. 11,686,737.
Application No. 16/868,447 is a continuation of application No. 15/785,205, filed on Oct. 16, 2017, now Pat. No. 10,677,804.
Application No. 15/785,205 is a continuation of application No. 12/637,730, filed on Dec. 14, 2009, now Pat. No. 9,791,458.
Application No. 12/637,730 is a continuation of application No. 12/300,118, filed on Jan. 1, 0001, now abandoned.
(60) Provisional application No. 60/798,712, filed on May 9, 2006.

(51) Int. Cl. G01N 33/68; G01N 33/564; A61P 19/02

(52) U.S. Cl. CPC G01N 33/6893; G01N 33/564; A61P 19/02; G01N 2800/102

(57) ABSTRACT

Methods and kits for diagnosing arthritis are provided. The methods may involve detection of 14-3-3 eta or gamma proteins in a sera or synovial fluid sample.

### CROSS-REFERENCE

This application a continuation of U.S. patent application Ser. No. 12/637,730, filed Dec. 14, 2009, which is a continuation of U.S. patent application Ser. No. 12/300,118, filed Nov. 6, 2008, which is a National Phase Entry of PCT Application No. PCT/CA2007/000817, filed May 9, 2007, which claims the benefit of U.S. Provisional Application No. 60/798,712, filed May 9, 2006, each of which is incorporated herein by reference in their entirety.

### SEQUENCE LISTING

The instant application contains a Sequence Listing which has been submitted in ASCII format via EFS-Web and is hereby incorporated by reference in its entirety. Said ASCII copy was created on Oct. 13, 2017, is named 177000-seq-list.txt and is 40,524 bytes in size.

### FIELD OF THE INVENTION

The invention relates to assays for selected protein isoforms that are diagnostic biomarkers for arthritis when those proteins are found in extracellular fluids.

### BACKGROUND OF THE INVENTION

Arthritis or arthralgia generally refers to inflammatory disorders of the joints of the body, and is usually accompanied by pain, swelling and stiffness. Arthritis may result from any of several causes including infection, trauma, degenerative disorders, metabolic disorders or disturbances or other unknown etiologies. Arthritis may be more specifically described as, for example, rheumatoid arthritis, osteoarthritis, bacterial or infectious arthritis. Arthritis may further accompany other identified disorders, including gout, ankylosing spondylitis, inflammatory bowel disease or psoriasis.

In normal joints, a small amount of synovial fluid (SF) lubricates cartilage and the synovium, and acts as a reservoir for solutes and a few resting mononuclear and synovial cells (3). During chronic inflammation, SF volume and the concentration of immune cells and soluble proteins increase (4).

For some forms of arthritis, such as rheumatoid arthritis (RA), the specific cause may not be known. RA is regarded as a “multifactorial threshold model”, in which many genetic and environmental influences must act on the same person in order for the disease to manifest (1). As a specific target is lacking, current therapies are primarily aimed at suppression of the inflammatory response (2). A hallmark of RA is synovial hyperplasia, characterized by fibroblast-like synoviocyte (FLS) proliferation and inflammatory cell infiltration into the subintima, or outer layer of the synovium (5). The FLS, which comprise about two-thirds of the synovium population, have a well-defined secretory system (5) and secrete large amounts of destructive matrix metalloproteases (MMPs) in RA (6), specifically MMP-1, 3, 8, 9, 10, 11 and 13 (7-11). Numerous researchers have shown that MMP-1 and MMP-3 play important roles in RA (12) and that the collagenase (MMP-1) is the most abundant (6). Both MMP-1 and MMP-3 are biomarkers that have been shown to have predictive validity for structural damage in RA. Local expression of MMPs in arthritis, especially MMP-1, is particularly prominent in the joint pannus adjacent to the site of cartilage and bone destruction (13). The collagenases, particularly MMP-1, cleave native collagen molecules at neutral pH, rendering the collagen susceptible to further enzymatic degradation (14).

Known factors that activate FLS to produce MMP-1 include pro-inflammatory cytokines such as interleukin-1 (IL-1) and tumour necrosis factor alpha (TNF-alpha), and both are involved in RA (15). IL-1alpha and TNF-alpha are capable of stimulating the production of other MMPs and stromelysins in synovial fibroblasts and chondrocytes in vitro (16). The interactions of FLS with TNF-alpha or IL-1 alpha from activated T cells induces expression of MMP-1 (17). T-cells can also activate FLS to produce an array of inflammatory mediators (18). This cyclical feedback loop between FLS and T cells and their respective cytokines lead to activation and proliferation of T cells and favours the persistent inflammation observed in RA (19-21). It has been suggested that therapeutic anti-TNF alpha antibodies neutralize TNF alpha and block the T cell activation that leads to this persistent state (22).

14-3-3 proteins are a family of dimeric proteins involved in a range of functions (23-24). There are seven mammalian 14-3-3 isoforms: beta (β), gamma (γ), epsilon (ε), eta (η), sigma (σ), tau (τ) and zeta (ζ). Since the discovery of the first 14-3-3 protein in 1967 (26), the members of the 14-3-3 protein family have been repeatedly re-discovered based on their new biological activities, primarily in signal transduction pathways. They have been identified as activators of tryptophan and tyrosine hydroxylase (27-28) and PKC inhibitors (29). Subsequent studies identified the 14-3-3 proteins as molecules that interact with PKCs, Raf family members and now more than 200 other intracellular proteins with critical biological functions (30-31) including cellular response to DNA damage and cell cycle regulation (32-34).

### SUMMARY OF THE INVENTION

The invention is based in part on the surprising discovery that particular isoforms of the 14-3-3 protein, 14-3-3 eta and gamma, are present at increased levels in the serum and synovial fluid of arthritis patients, compared to normal patients.

In accordance with one aspect of the invention, there is provided a method for predicting responsiveness of a subject to a therapeutic regimen, the subject having, or suspected of having arthritis, the method comprising determining a presence, absence, amount or relative levels of a protein 14-3-3 eta or gamma in a sample, wherein the presence, absence, amount or relative level of the isoform is indicative of a sensitivity of the subject's arthritis to the therapeutic regimen.

In accordance with another aspect of the invention, there is provided a kit for detecting a 14-3-3 eta or gamma protein in a patient sample, such as a sample of sera or synovial fluid from a patient having, or at risk of having, an arthritis. The kit may comprise at least one antibody specific for detecting at least one of these isoforms of the 14-3-3 protein. The kit may further comprise at least one antibody specific for detecting at least one matrix metalloproteinase.

In accordance with another aspect of the invention, there is provided a method for selecting a group of subjects for determining the efficacy of a therapeutic regimen known or suspected of being useful for the treatment of arthritis, the method comprising detecting a presence, absence, amount or relative levels of a 14-3-3 eta or gamma protein in a patient sera or synovial fluid sample, wherein said presence, absence, amount or relative level of one or more of these isoforms of the 14-3-3 protein in that fluid is indicative of a sensitivity of the subject's arthritis to the therapeutic regimen.

In accordance with another aspect of the invention, there is provided a method of treating arthritis in a mammalian subject in need thereof, the method comprising administering to the subject a therapeutic regimen, wherein the presence, absence, amount or relative level of a 14-3-3 eta or gamma protein isoform in sera or synovial fluid from that subject is indicative of sensitivity to the therapeutic regimen.

In accordance with another aspect of the invention, there is provided a method of treating arthritis in a mammalian subject in need thereof, the method comprising: selecting a subject having a presence, absence, amount or relative level of 14-3-3 eta or gamma protein in sera or synovial fluid that is indicative of sensitivity to a therapeutic regimen; and administering to the subject the therapeutic regimen.

In accordance with another aspect of the invention, there is provided a method of identifying a mammalian subject with an increased sensitivity to a therapeutic regimen for treating arthritis, comprising the step of screening a population of subjects for the presence, absence, amount or relative level of 14-3-3 eta or gamma protein in sera or synovial fluid, and identifying subjects sensitive to the therapeutic regimen based at least in part on the presence, absence, amount or relative level of the protein in the fluid.

In selected embodiments, the invention may involve assays for other arthritis markers in the sera or synovial fluids of subjects, in conjunction with assays for the presence, absence, amount or relative level of 14-3-3 eta or gamma proteins. For example, assays of the invention may additionally involve determining the presence, absence, amount or relative levels of one or more matrix metalloproteinases, such as MMP-1 or MMP-3, in sera or synovial fluid samples.

### BRIEF DESCRIPTION OF THE DRAWINGS

In drawings which illustrate embodiments of the invention,

FIG. 1 Detection of various isoforms of 14-3-3 in the synovial fluid (SF) and serum (PS) of arthritic patients by western blot. Keratinocyte lysates (K) was used as a positive control.

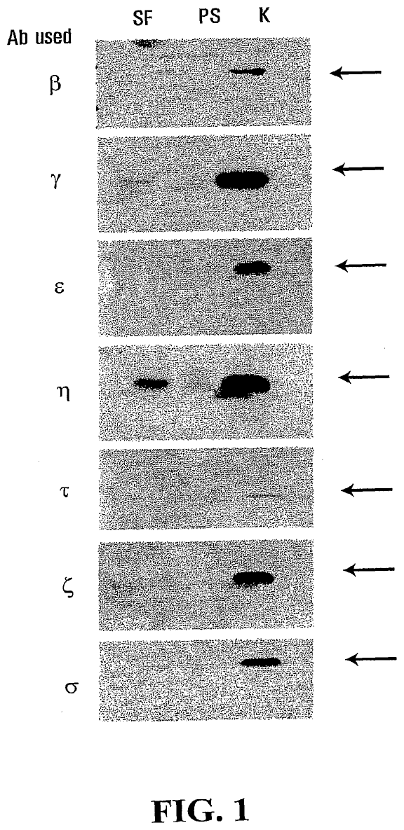

FIG. 2. Detection of 14-3-3 η in the synovial fluid of 17 arthritic patients. Synovial fluid samples (2 μl/lane) were taken from 17 RA patients who had active synovitis and analyzed by western blot, using an isoform-specific antibody for the 14-3-3 η isoform. Keratinocyte lysate (K) was used as a positive control. Levels of 14-3-3 η vary in the patient population surveyed, but is reliably detected in all synovial fluid samples.

FIG. 3 14-3-3 η, MMP-1 and MMP-3 expression in patient sera and synovial fluid. 12 patients' matched synovial fluid and serum samples examined by western blot. Keratinocyte cell lysate (K) was used as a positive control. SF: synovial fluid; PS: patient serum; MMP-1: matrix metalloproteinase 1; MMP-3: matrix metalloproteinase 3.

FIG. 4 Detection and comparison of the levels of 14-3-3 n and γ in 9 matched patient serum and synovial fluid samples. Patients' synovial fluid or serum (2 μl/lane) was analyzed by western blot using anti 14-3-3 η or γ antibody. Keratinocyte cell lysate (K) was used as a positive control. SF, synovial fluid; PS, patient serum.

FIG. 5 Levels of 14-3-3 η in matched patient serum and synovial fluid samples were quantified by densitometry and depicted in lower panel. Solid bars show the level of 14-3-3 η in serum normalized to a synovial fluid sample from the same patient (open bars).

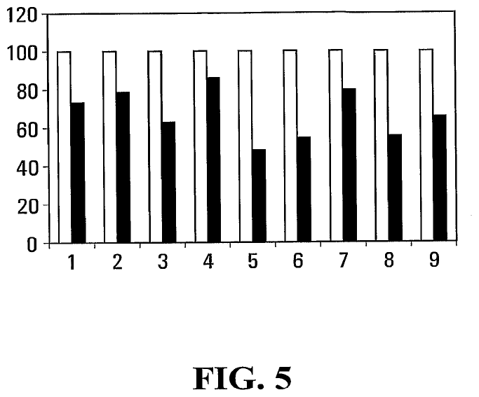

FIG. 6A-B Detection of 14-3-3 η, γ, MMP-1 and MMP-3 in different volumes of normal and patients sera.

A) Pooled samples of 12 normal or patient sera were prepared and a range of volumes (0.1-2.0 μl/lane) were analyzed by western blot, using specific antibodies for 14-3-3 η, γ, MMP-1 or MMP-3. 2 μl of a pooled of synovial fluid (SF) from affected patients was included as a positive control. B) Recombinant 14-3-3 η isoform (0.01-2.0 μg/lane) was analyzed by western blot in parallel with 2 μl of normal (NS) or patient serum (PS).

FIG. 7 Illustrates detection of 14-3-3 η before and after anti-TNF therapy:

Levels of 14-3-3 eta protein in 4 ml of serum samples from RA patients before (U) and after anti-TNF-a (T) Treatment. N=negative control which is 4 ml of a pooled serum sample prepared from 12 serum samples taken from 12 healthy individuals. P=positive control which is 4 mg of keratinocyte cell lysate total protein.

### DETAILED DESCRIPTION

A “Disease Activity Score” (DAS) refers to a measure of the activity or state of rheumatoid arthritis in a patient. DAS is one of several standards or scores used in clinical practice. A calculation of a DAS may include the following parameters: Number of joints tender to the touch (TEN), number of swollen joints (SW), erythrocyte sedimentation rate (ESR) and patient assessment of disease activity (VAS). Alternatively, a DAS may include C-reactive protein marker assessment (CRP) (Skogh T et al 2003. Ann Rheum Dis 62:681-682).

A patient or test subject, as used herein, includes a human patient undergoing, or about to undergo, treatment for arthritis. A test subject includes non-human mammals undergoing, or about to undergo, treatment for arthritis. In the case of a test subject, the arthritis may be deliberately induced or implanted, or may develop spontaneously. The patient or test subject may have been previously diagnosed using methods described herein, for example, or other diagnostic methods known in the art, or may be selected as part of general population (a ‘control’ patient or ‘control’ test subject). Patients and test subjects, whether control or not, may be generally referred to as a subject. Patients may be selected or differentiated on the basis of disease severity, gender, age, or suitability for a particular treatment or assay method.

As used herein, an ‘isoform’ refers to any two or more of functionally similar proteins that have a similar but not identical amino acid sequence and are either encoded by different genes or by RNA transcripts from the same gene which have had different exons removed.

It will be appreciated by a person of skill in the art that the numerical designations of the positions of nucleotides or amino acids within a sequence are relative to the specific sequence. Also, the same positions may be assigned different numerical designations depending on the way in which the sequence is numbered and the sequence chosen. Furthermore, sequence variations such as insertions or deletions, may change the relative position and subsequently the numerical designations of particular nucleotides or amino acids at or around a particular site.

The terms ‘peptide’, ‘polypeptide’ and protein’ may be used interchangeably, and refer to a compound comprised of at least two amino acid residues covalently linked by peptide bonds or modified peptide bonds, for example peptide isosteres (modified peptide bonds) that may provide additional desired properties to the peptide, such as increased half-life. A peptide may comprise at least two amino acids. The amino acids comprising a peptide or protein described herein may also be modified either by natural processes, such as posttranslational processing, or by chemical modification techniques which are well known in the art. Modifications can occur anywhere in a peptide, including the peptide backbone, the amino acid side-chains and the amino or carboxyl termini. It is understood that the same type of modification may be present in the same or varying degrees at several sites in a given peptide.

Nomenclature used to describe the peptide compounds of the present invention follows the conventional practice where the amino group is presented to the left and the carboxy group to the right of each amino acid residue. In the sequences representing selected specific embodiments of the present invention, the amino- and carboxy-terminal groups, although not specifically shown, will be understood to be in the form they would assume at physiologic pH values, unless otherwise specified. In the amino acid structure formulae, each residue may be generally represented by a one-letter or three-letter designation, corresponding to the trivial name of the amino acid.

The term ‘antibody’ as used herein includes polyclonal and monoclonal antibodies, chimeric, single chain, or humanized antibodies, as well as Fab or F(ab)² fragments, including the products of an Fab or other immunoglobulin expression library. Methods of making such antibodies or fragments are known in the art and may be found in, for example HARLOW, E and LANE D. Antibodies: A Laboratory Manual. 1988. Cold Spring Harbor Laboratory Press. Selection or identification of specific peptides for use as epitopes for production of antibodies that differentiate between proteins, or isoforms of proteins may be made using sequence comparisons—one of skill in the art will be able to identify suitable peptide or protein sequences that may be useful for producing antibodies with the desired selectivities. Examples of sequences that may be useful to one of skill may include SEQ ID NOs: 1-7.

As used herein, ‘arthritis’ or ‘arthralgia’ refer to an inflammatory disorder of the joints of the body. Pain, swelling, stiffness and difficulty of movement are frequently associated with arthritis diseases. Arthritis may result from any of several causes including infection, trauma, degenerative disorders, metabolic disorders or disturbances or other unknown etiologies. Arthritis may be more specifically described as, for example, rheumatoid arthritis, osteoarthritis, bacterial or infectious arthritis. Arthritis may further accompany other identified disorders, including gout, ankylosing spondylitis, inflammatory bowel disease or psoriasis.

14-3-3 proteins, particularly the eta and gamma isoforms, may be readily detected in synovial fluid or serum of patients affected with arthritis, for example rheumatoid arthritis. In one embodiment of the invention, detection of these signal transduction proteins in the site of inflammation may have application in early or more simplified diagnosis of arthritis, or differentiation between the various types of arthritis in a patient. Alternatively, the presence or relative levels of isoforms of 14-3-3 proteins may be a prognostic indicator of early-stage arthritis, before it progresses to a debilitating form. An advantage of early prognosis or diagnosis is earlier implementation of a treatment regimen. Alternatively, the presence or relative levels of isoforms of 14-3-3 in a patient sample may be useful to determine patient suitability for a particular treatment regimen.

Treatment regimens for various types of arthritis are known in the art. Therapeutic approaches to arthritis may for example be generally characterised as disease modifying therapy for arthritis, or remittive therapies. For example, a patient diagnosed with rheumatoid arthritis may be prescribed non-steroidal anti-inflammatory medications (NSAIDs) initially, to ease the discomfort and reduce the inflammation. Other treatment regimens may include, for example cyclooxygenase 2 specific inhibitors (CSIs), glucocorticoids, disease-modifying anti-rheumatic drugs (DMARDs), anti-TNF alpha neutralizing agents or immunosuppressive or cytotoxic drugs. Details on dosage or examples of particular drugs will be known to those of skill in the art, and may be found in, for example Harrison's Principles of Internal Medicine 15^{th} ed. BRAUNWALD et al eds. McGraw-Hill or “The Pharmacological basis of therapeutics”, 10^{th} edition. HARDMAN HG., LIMBIRD LE. editors. McGraw-Hill, New York, and in “Clinical Oncology”, 3^{rd} edition. Churchill Livingstone/Elsevier Press, 2004. ABELOFF, MD. editor.

In another embodiment of the invention, the presence or relative levels of 14-3-3 eta or gamma proteins may correlate with the presence or relative levels of other proteins in the patient sample, for example matrix metalloproteinases (MMPs), such as MMP-1 or MMP-3. MMPs are zinc-binding endopeptidases that degrade components of the extracellular matrix. MMPs have different substrate specificities and are encoded by different genes. At least 25 different MMPs have been identified. Detection of 14-3-3 eta or gamma proteins in combination with at least one MMP in a patient sample may have application in early or more simplified diagnosis of arthritis, or differentiation between the various types of arthritis in a patient. Alternatively, the presence or relative levels of eta or gamma isoforms of 14-3-3 proteins in combination with at least one MMP in a patient sample may be a prognostic indicator of early-stage arthritis, before the arthritis progresses to a debilitating form. An advantage of early prognosis or diagnosis may include earlier implementation of a treatment regimen. Alternatively, the presence or relative levels of eta or gamma isoforms of 14-3-3 in combination with at least one MMP in a patient sample may be useful to determine patient suitability for a particular treatment regimen.

In another embodiment of the invention, a kit for detecting the presence of 14-3-3 eta or gamma proteins or particular MMPs in a patient sample, the kit being suitable for use in providing a diagnostic or prognostic result suitable for diagnosing or differentiating various types of arthritis. A kit may include, for example, antibodies specific for eta or gamma isoforms of 14-3-3 proteins. Such a kit may further include antibodies specific for particular MMPs. The kit may further include other reagents necessary for the detection of 14-3-3 eta or gamma or MMPs immunologically, such as labelled secondary antibodies, chromogenic or fluorogenic reagents, polymerization agents and/or instructions for using the kit for diagnostic or prognostic purposes.

### General Methods

Once a subject is identified as being at risk for developing or having arthritis, information useful for assessing the disease state of the diagnosed arthritis, response to a therapeutic treatment regimen for arthritis, or prognosis of arthritis, or the type of arthritis may be obtained from the patient or test subject. Various methods for obtaining biological samples from a subject that contain protein or peptides that may be useful as biomarkers are known in the art. For example, tissue samples may be obtained by curettage, needle aspiration biopsy or needle (core) biopsy, incisional biopsy for sampling a tumor, or excisional biopsy, which may entail total removal of the tissue of interest. Alternatively, other bodily samples that contain genetic material, such as synovial fluid, hair, sputum, urine, stool, semen, plasma, serum or blood may be collected using methods known in the art.

The presence of specific proteins or peptides in a biological sample, or the relative levels of specific proteins or peptides in a biological sample may be detected by any of several methods known in the art. Examples of such methods include mass spectroscopy, immunological-based techniques such as western blotting, ELISA, immunohistochemistry, FACS, surface plasmon resonance or chromatography. Methods for these and other techniques may be found in, for example AUSUBEL et al., Current Protocols in Molecular Biology, John Wiley & Sons, New York, N.Y., 1998: ABELOFF, Clinical Oncology, 3^{rd} edition. Churchill Livingstone/Elsevier Press, 2004; HARLOW, E and LANE D. Antibodies: A Laboratory Manual. 1988. Cold Spring Harbor Laboratory Press; SAMBROOK J and RUSSELL DW. Molecular cloning: A Laboratory Manual 2001 Cold Spring Harbor Laboratory Press; Harrison's Principles of Internal Medicine 15^{th} ed. BRAUNWALD et al eds. McGraw-Hill. 14-3-3 detection methods are described for example in WO 99/46401, US 2005/9094, WO 97/38315 and WO 97/33601, all of which are incorporated herein be reference.

### Western Blotting

Synovial fluid or serum (2 μl of each) was subjected to SDS-PAGE analysis with 12% (wt/vol) acrylamide gel, and electrotransferred onto PVDF membranes (Millipore Corporation). Non-specific proteins on membranes were blocked in 5% skim milk powder in PBS-0.1% Tween-20 overnight. Immunoblotting was performed using 2 μg/ml of 7 isoforms specific rabbit anti-human 14-3-3 polyclonal antibodies (Martin H, Patel Y, Jones D, Howell S, Robinson K and Aitken A 1993. Antibodies against the major brain isoforms of 14-3-3 protein. An antibody specific for the N-acetylated amino-terminus of a protein. FEBS Letters. 331:296-303). The membranes were then incubated with the appropriate secondary horseradish peroxidise conjugated anti-rabbit IgG (Sigma, St Louis, USA) or anti-mouse IgG (Bio-Rad Laboratories, Hercules, USA) antibodies (1:2500 dilution). Immunoreactive proteins were then visualized using the ECL+plus western blotting detection system (Amersham Biosciences, Buckinghamshire, England). Keratinocyte cell lysate (K) was used as a positive control. SF: synovial fluid; PS: patient serum.

### Patient Samples

Synovial fluid was obtained from the knee joints of patients with active synovitis prior to the institution of anti-TNF therapeutics. All patients had a DAS score >6.0. Matched blood samples were obtained by standard venipuncture procedures. The clot was removed by centrifugation.

### Recombinant 14-3-3 Eta

cDNA for keratinocyte-derived 14-3-3 eta was prepared from total RNA extracted from human keratinocytes, cloned and expressed in E. coli, and affinity purified, following the methods described in Ghahary et al 2004 J Invest Dermatol 122:1188-1197. Primers used for PCR amplification of the 14-3-3 eta cDNA were

SEQ ID NO: 15
(GCGAATTCCTGCAGCGGGCGCGGCTGGCCGA)
and
SEQ ID NO: 16
(GCTCGAGCCTGAAGGATCTTCAGTTGCCTTC).

### Example 1

### 14-3-3 Expression in Synovial Fluid and Serum of RA Affected Patients

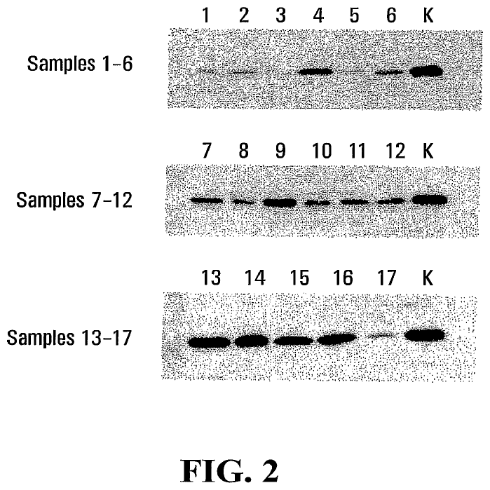

The levels of the different isoforms of 14-3-3 proteins—β, γ, ε, η, τ σ and ζ—in pooled patient synovial fluid (SF) and serum (PS) samples were analyzed by western analysis using keratinocyte cell lysate (K) as a positive control. Only the n and γ isoforms were detected in SF samples, and stained with greater intensity compared to PS. Articular joint synovial fluid samples from 17 RA patients who presented with active synovitis, but had not yet received anti-TNF therapies also exhibited consistent expression of then isoform of 14-3-3 (FIG. 2). All patients had a disease activity score (DAS) greater than 6.0.

### Example 2

### MMP Expression in Patient Synovial Fluid Serum

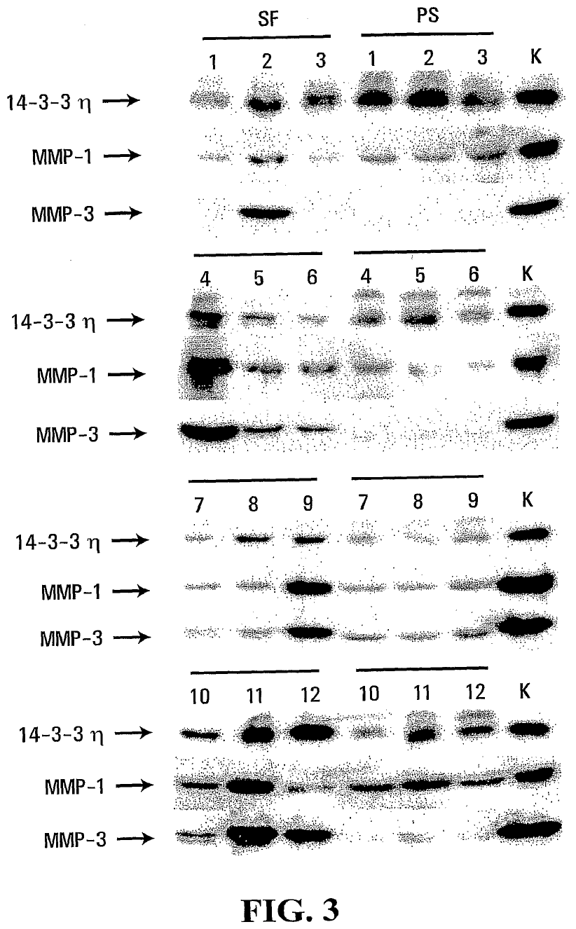

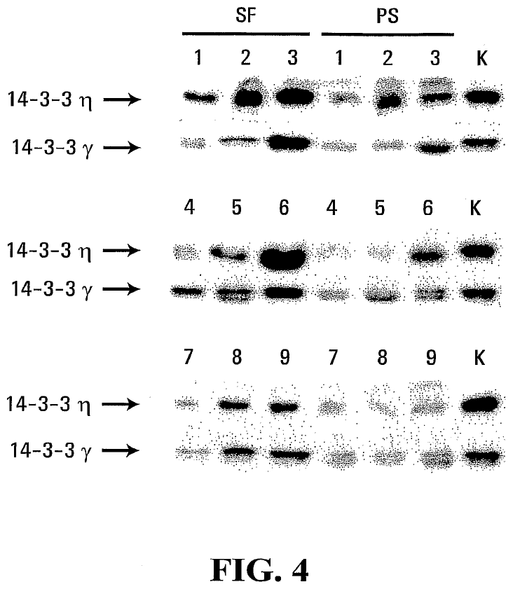

To determine if these variations were correlated to those of MMP-1 and MMP-3 in the same synovial samples, a total of 12 RA synovial fluid samples and their matched serum samples were simultaneously evaluated for 14-3-3 η and γ as well as for MMP-1 and MMP-3 proteins (FIG. 3). 14-3-3 η was detected in all samples. MMP-1 was detected in all samples, both SF and PS, while MMP-3 was more variable in the levels detected. The 14-3-3 γ isoform was also detected in patient synovial fluid and serum samples (FIG. 4, 5).

The expression of MMP-1 and MMP-3 demonstrate significant correlation with the expression of the 14-3-3 η and γ isoforms in both synovial fluid and serum (Table 1).

TABLE 1
Correlation of MMP and 14-3-3 protein levels in serum and synovial fluid.

| | 14-3-3 η | 14-3-3 η | 14-3-3 γ | 14-3-3 γ |
|---|---|---|---|---|
| | serum | synovium | serum | synovium |
| MMP-1 | r = 0.62; | r = 0.83; | r = 0.77; | r = 0.65; |
| | p = 0.02 | p = 0.03 | p = 0.02 | p = 0.03 |
| MMP-3 | r = 0.68; | r = 0.77; | r = 0.80; | r = 0.76; |
| | p = 0.01 | p = 0.003 | p = 0.03 | p = 0.04 |

### Example 3

### Sensitivity of Western Blot Detection of 14-3-3 Protein in Patient Serum and Synovial Fluid Samples

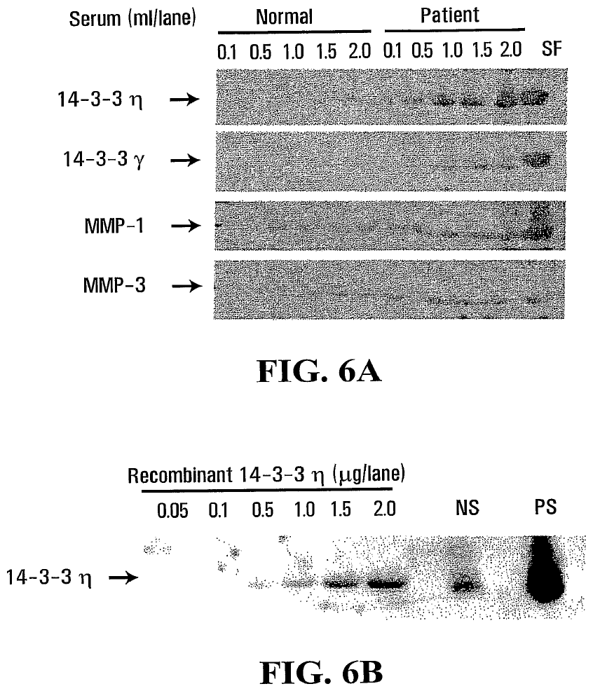

To determine the detection level of 14-3-3 η in synovial fluid and serum samples, samples from 12 RA-affected or normal patients were pooled, and limiting dilutions of the pooled samples were analyzed by western blot. 14-3-3 η was detectable over a range of dilutions—as low as 0.1 μl effective volume of synovial fluid and 1.0 μl effective volume of serum (FIG. 6A).

2 μl of pooled normal serum (NS) or patient serum (PS) was run alongside known concentrations of recombinant 14-3-3 η, ranging from 0.05-2.0 μg. The 2 μl volume of NS and PS samples was estimated to have approximately 1-1.5 and 15-20 μg of 14-3-3 η, respectively (FIG. 6B). This suggests that the level of 14-3-3 η occurs in about a 10-fold excess in the serum of RA affected patients, compared to normal patients.

### Example 4

### Detection of 14-3-3 η Before and After Anti-TNF Therapy

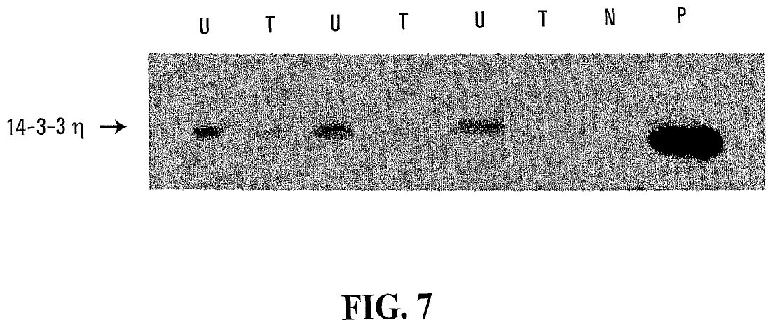

FIG. 7 illustrates the sequential detection of levels of 14-3-3 eta protein in 4 ml of serum samples from RA patients before (U) and after anti-TNF-a (T) Treatment. N=negative control which is 4 ml of a pooled serum sample prepared from 12 serum samples taken from 12 healthy individuals. P=positive control which is 4 mg of keratinocyte cell lysate total protein.

### REFERENCES

The following documents are incorporated herein by reference:
1. Harris E D Jr., History and Epidemiology of Rheumatoid Arthritis: How long has it affected us, and who is at risk? In: Rheumatoid Arthritis. Philadelphia: W.B. Saunders Company, 1997: 21-27.
2. Harris E D Jr., Introduction. In: Rheumatoid Arthritis. Philadelphia: W.B. Saunders Company, 1997: xix-xxiii.
3. Harris E D Jr., Rheumatoid Synovium: Complex, and More Than the Sum of its Parts. In: Rheumatoid Arthritis. Philadelphia: W.B. Saunders Company, 1997: 127-149.
5. Firestein G S. (1997). Rheumatoid synovitis and pannus. In: J. H. Klippel and P. A. Dieppe, Editors, Rheumatology, Mosby, London, pp. 5/13.1-5/13.5, 1997.
6. Pap T, Shigeyama Y, Kuchen S. (2000). Arthritis Rheum. 43: 1226-1232.
7. Tolboom T C A, Pieterman E, van der Laan W E. Ann. Rheum. Dis. 61: 975-980, 2002.
8. Sorsa T, Konttinen Y T, Lindy O. Arthritis Rheum. 22: 44-53, 1992.
9. Lindy O, Konttinen Y T, Sorsa T. Arthritis Rheum. 40:1391-1399, 1997.
10. Ahrens D, Koch A E, Pope R M. Arthritis Rheum. 39:1576-1587, 1996.
11. Smeets T J M, Dayer J M, Karan M C. Arthritis Rheum. 43:270-274, 2000.
12. Poole A R; Cartilage in health and disease. In: Koopman W J. Ed. Arthritis and Allied conditions. A textbook of rheumatology. 14th ed. Baltimore: Williams and Wilikins, 2001: 226-284.
13. Konttinen Y T, Ceponis A, Takagi M, Ainola M, Sorsa T, Sutinen M, et al. Matrix Biol. 17:585-601, 1998.
14. Katrib. A, McNeil H P, Youssef P P: Inflamm. Res. 51: 170-175, 2002.
15. Harris E D Jr., Cytokines, Lymphokines, Growth Factors, and Chemokines. In: Rheumatoid Arthritis. Philadelphia: W.B. Saunders Company, 1997: 105-125.
16. Jasser, M. Z., Mitchell P. G. and Cheung, H. S.: induction of stomelysin-1 and collagenases synthesis in fibrochondrocytes by TNF-alpha. Matrix Biology 14: 241, 1994.
17. Burger D, Rezzonico R, Li J M, Modoux C, Pierce R A, Welgus H G, Dayer J M. Arthritis Rheum. 41(10):1748-59, 1998
18. Y. Yamamura, R. Gupta, Y. Mont, X. He, R. Pai, J. Endres, A. Freiberg, K. Chung and D. A. Fox. J. Immunol. 166 (2001), pp. 2270-2275
19. Miranda-Carus M E, Balsa A, Benito-Miguel M, Perez de Ayala C, Martin-Mola E. J. Immunol. 173:1463-1476, 2004
20. Cho M L, Yoon C H, Hwang C Y. Arthritis Rheum. 50:776-784, 2004
21. Bombara M P, Webb D L, Conrad P. J. Leukocyte Biol. 54: 399-406, 1993.
22. McInnes I B, Leung B P, Liew F Y. Arthritis Res. 2(5):374-8.34, 2000.
23. FU H, Subramanian R R, Masters S C: Annu Rev Pharmacol Toxicol 40:617-647, 2000.
24. Hsich G, Kenney K, Gibbs C J, Lee K H, Harrington M G: N Engl J Med 335:924-30, 1996
25. Wilker E, Yaffe M B: J Mol Cell Cardiol 37: 633-642, 2004.
26. Moore et al. 1967.
27. Ichimura T, Isobe T, Okuyama T, Yamauchi T, Fujisawa H (1987) FEBS Lett. 219:79-82.
28. Ichimura T, Isobe T, Okuyama T, Takahashi N, Araki K, Kuwano R, Akahashi Y (1988). Proc Natl Acad Sci USA, 85:7084-8.
29. Toker A, Ellis C A, Sellers L A, Atiken A 1990. Eur J Biochem 191:421-429.
30 Craparo A, Freund R, Gustafson T (1997). J Biol Chem 272:11663-69.
31. Yaffe M B (2002). FEBS Lett 513(1):53-57.
32. Hermeking H, Lengauer C, Polyak K, He T C, Zhang L, Thiagalingam S, Kinzler K W, Volgelstein B. Mol Cell 1:3-11, 1997.
33. Chan T A, Hermeking H, Lengauer C, Kinzler K W, Volgelstein B. Nature 401:616-620, 1999.
34. Laronga C, Yang H Y, Neal C, Lee M H (2000). J. Biol. Chem. 275:23106-23112.
35. Boston P F, Jackson P, Thompson R J. J. Neurochem. 38, 1475-82, 1982.
36. Katz A B, Taichman, L B. J Invest Dermatol 112:818-821, 1999.
37. Ghahary A, Karimi-Busheri F, Marcoux Y, Li Y, Tredget E E, Kilani R T, Li L, Zheng J, Karami A, Keller B, Weinfeld M. J. Invest. Dermatol. 122(5): 1188-1197, 2004.
38. Ghaffari A, Li Y, Karami A, Ghaffari M, Tredget E E, Ghahary A. J. Cell. Biochem. January 2006.

While specific embodiments of the invention have been described and illustrated, such embodiments should be considered illustrative of the invention only and not as limiting the invention as construed in accordance with the accompanying claims.

1-13. (canceled)

14. A method of detecting the eta isoform of the 14-3-3 protein in a patient, said method comprising:
a. obtaining a test sample from a human patient at risk or suspected of having an inflammatory disorder of the joints of the body, wherein the test sample is selected from the group consisting of blood, serum, plasma, and synovial fluid; and
b. detecting whether 14-3-3 eta protein is present in the test sample by mass spectrometry.

15. The method of claim 14, wherein said 14-3-3 eta isoform comprises the amino acid sequence of SEQ ID NO: 3.

16. The method of claim 14, further comprising detecting at least one additional protein in said test sample.

17. The method of claim 16, wherein said at least one additional protein is a matrix metalloproteinase.

18. The method according to claim 14, wherein said sample is serum.

19. A method of detecting the eta isoform of the 14-3-3 protein in a patient, said method comprising:
a. obtaining a test sample from a human patient having pain, swelling, and stiffness in the joints of the body, wherein the test sample is selected from the group consisting of blood, serum, plasma, and synovial fluid; and
b. detecting whether 14-3-3 eta protein is present in the test sample by mass spectrometry.

20. The method of claim 19, wherein said 14-3-3 eta isoform comprises the amino acid sequence of SEQ ID NO: 3.

21. The method of claim 19, further comprising detecting at least one additional protein in said test sample.

22. The method of claim 21, wherein said at least one additional protein is a matrix metalloproteinase.

23. The method according to claim 19, wherein said sample is serum.

24. A method of detecting the eta isoform of the 14-3-3 protein in a patient, said method comprising:
a. obtaining a test sample from a human patient at risk or suspected of having arthritis, wherein the test sample is selected from the group consisting of blood, serum, plasma, and synovial fluid; and
b. detecting whether 14-3-3 eta protein is present in the test sample by mass spectrometry.

25. The method of claim 24, wherein said 14-3-3 eta isoform comprises the amino acid sequence of SEQ ID NO: 3.

26. The method of claim 24, further comprising detecting at least one additional protein in said test sample.

27. The method of claim 26, wherein said at least one additional protein is a matrix metalloproteinase.

28. The method according to claim 24, wherein said sample is serum.

29. The method of claim 24, wherein the arthritis is rheumatoid arthritis.

30. The method of claim 24, wherein the arthritis is early stage arthritis.
